(19) United States
(12) Patent Application Publication
(10) Pub. No.: US 2008/0038350 A1
(43) Pub. Date: Feb. 14, 2008

(54) LOW-DOSAGE PERORAL MEDICATION FOR CONTRACEPTION CONTAINING CRYSTALLINE DIENOGEST AND ETHINYL ESTRADIOL

(76) Inventors: Hagen Gerecke, Jena (DE); Sabine Fricke, Jena (DE); Ralf Ladwig, Jena (DE); Herbert Wiesinger, Berlin (DE); Alexander Buske, Jena (DE)

Correspondence Address: STRIKER, STRIKER & STENBY, 103 EAST NECK ROAD, HUNTINGTON, NY, 11743, US

(21) Appl. No.: 11/762,207

(22) Filed: Jun. 13, 2007

Related U.S. Application Data
(60) Provisional application No. 60/836,914, filed on Aug. 10, 2006.

(51) Int. Cl. A61K 31/56; A61K 9/28; A61P 15/18

(52) U.S. Cl. 424/474; 514/171

(57) ABSTRACT

The peroral medication for prevention of conception contains as one active ingredient crystalline 17α-cyanomethyl-17β-hydroxyestra-4,9-dien-3-one (dienogest) at a daily dosage equal to or less than 2.0 mg and as another active ingredient 17α-ethinyl estradiol at a daily dosage of less than 0.030 mg, together with one or more pharmaceutically acceptable carriers. The active ingredient dienogest is contained in the medication in crystalline form with an average particle size of preferably 25 to 70 μm. The other active ingredient ethinyl estradiol is incorporated during granulation in micronized form or by spraying an ethanolic solution containing it.

### CROSS-REFERENCE

U.S. Provisional Application No. 60/836,914, filed Aug. 10, 2006, also discloses the invention described and claimed herein and provides a basis for a claim of priority for that invention under 35 U.S.C. 119(e).

### BACKGROUND OF THE INVENTION

1. The Field of the Invention

The subject matter of the present invention is a peroral medication for contraception which contains 2.0 mg or less of crystalline 17α-cyanomethyl-17β-hydroxyestra-4,9-dien-3-one (crystalline dienogest) and less than 0.030 mg of 17α-ethinyl estradiol (ethinyl estradiol) and wherein the crystalline active ingredient dienogest has an average particle size of 25 to 270 μm.

2. Related Art

Oral conception-preventing agents consisting of a gestagen component and an estrogen component first came on the market in the early 1960's. Three essential properties characterize the profile of the “preventive pill”: contraceptive reliability, very good cycle control, and a minimum of side effects.

Since the introduction of hormonal contraceptives, research has been directed toward the possibility of creating medications which, while retaining good contraceptive reliability and cycle control, reduced undesirable side effects. These side effects included arterial and venous thrombosis and effects on carbohydrate and fat metabolism, which are caused by a higher gestagen and estrogen content than needed for the prevention of contraception. WO 98/004269 disclosed, among other things, oral administration of a combination of 250 μg-4 mg of dienogest and 10 μg-20 μg of ethinyl estradiol for contraception. To attain the essential reduction in the total amount of the contraceptive steroids administered per cycle while retaining good cycle control, the low-dosage gestagen/estrogen combination was administered for 23 to 25 days of a 28-day menstrual cycle. The patent, however, does not disclose any results or information that would indicate that the inventive idea actually leads to success and which form of steroid release should be strived for.

It is also known that it is imperative to make sure that low-dosage oral contraceptives are taken every day in the same time period. If this time period is not observed, the effective active ingredient concentration required for oral contraception is not reached and oral contraception is no longer ensured. This means that the user must be highly aware of her intake cycle and must follow it with great care.

It is also known from the technical and patent literature that particle size exerts an effect on the pharmacokinetic properties of an active ingredient. In the following, grain size may indicate particle size, and content uniformity may also indicate homogeneity of active ingredient distribution.

Bauer, et al, in “Lehrbuch der pharmazeutischen Technologie” [Textbook of Pharmaceutical Technology], 6th edition, Stuttgart, Wissenschaftliche Verlagsgsellschaft [publisher] 1999, report that the administration of a single dosage of digoxin with an average particle size of 3.7 μm produced a plasma level that was nearly twice as high as that found after the administration of a digoxin powder with an average particle size of 22 μm.

WO 2003 091272 discloses that low-dosage formulations require homogeneous active ingredient distribution (CUT) and appropriate dissolution kinetics. To this end, coarse crystals of an active ingredient are micronized in a jet mill in accordance with conventional technology. This affords an average particle size from 1.5 to 5 μm. WO 2003 091272 also indicates that particle sizes from 3 to 25 μm and preferably from 7 to 15 μm may be sufficient for low-dosage formulations. Explicit statements to this effect are not provided.

### SUMMARY OF THE INVENTION

It is an object of the present invention to reduce the steroid load or steroid content of conventional dienogest/ethinyl estradiol active ingredient combinations for oral contraception, while ensuring a contraceptive efficacy similar to that of conventional oral contraceptives.

According to the present invention, this objective and others, which will be made more apparent hereinafter, are attained by a solid peroral contraceptive medication containing, as active ingredient combination, 2.0 mg or less of crystalline dienogest and less than 0.030 mg of ethinyl estradiol, the crystalline active ingredient dienogest contained therein having an average particle size from 25 or 30 to 270 μm, advantageously 25 to 70 μm.

Advantageously, the particle size of the crystalline active ingredient dienogest in the medication of the invention is from 40 to 65 μm.

The active ingredient combination in the medication of the invention can also contain from 0.75 to 2.0 mg of crystalline dienogest and from 0.015 to 0.020 mg of ethinyl estradiol. The active ingredient ethinyl estradiol is sprayed on during granulation in the form of an ethanolic solution or is used in micronized form, preferably in the tablet matrix/tablet core.

The crystals of the crystalline active ingredient dienogest having a certain particle size are obtained from a supersaturated solution of the steroid, which is subjected to wet grinding during the crystallization. The primary particle suspension is obtained in this manner.

Other methods for producing crystalline dienogest with the desired particle size are also conceivable. They include grinding, sifting, or screening coarse materials.

The peroral medication of the invention can be a tablet, a tablet with a film coating (coated tablet), or a tablet with a sugar-containing covering (sugar-coated tablet). The peroral medications of the invention also include hard gelatin capsules, soft gelatin capsules with an oily or aqueous suspension as filling material, and other peroral suspensions. Pharmaceutically acceptable carriers can be included.

The release of the active ingredients or the leaching thereof from the tablet matrix/tablet core is measured by a dissolution test using water at 37° C. as the dissolution medium and a rotation speed of 50 rpm.

The measurement is carried out in accordance with the European Pharmacopeia using a blade agitator apparatus and 1000 mL of water.

A preferred medication of the invention is one for which the number of daily dosage units containing a combination of dienogest and ethinyl estradiol is 21, 22, 23, 24, 25, or 26 and the number of daily dosage units containing no active ingredient is 7, 6, 5, 4, 3, or 2 in a 28-day menstrual cycle.

Surprisingly, we have found that the use of the crystalline dienogest with an average particle size of 25 to 70 μm as the active ingredient in the matrix of the peroral medication makes it possible to use a low dosage of the dienogest/ethinyl estradiol active ingredient combination and associated therewith a reduction in the total steroid load, while retaining the same contraceptive efficacy as that of the conventional oral contraceptives that contain dienogest and ethinyl estradiol. Thus conception is reliably prevented when the medication according to the invention is administered. The medication can be taken, for example, in the morning or in the evening without necessarily having to observe an exact time for intake of the medication. Approximately the same time of day, however, should still be observed.

The bioavailability of the pharmaceutical active ingredients can also be determined from a plasma concentration curve as the area under the curve (AUC).

Surprisingly, we have also found that it is possible to reduce the total steroid load (reduction in the steroid load of dienogest as well as the reduction in the steroid load of ethinyl estradiol) with the peroral medication of the invention using a single-phase active ingredient release.

The pharmaceutical requirements for uniformity of the active ingredient distribution are determined by the parameters of the particle size distribution.

The particle size distribution of crystalline dienogest is characterized by a median of the particle size distribution X₅₀ (Sucker, H., et al., “Pharmazeutische Technologie” [Pharmaceutical Technology], page 16f, Georg Thieme Verlag Stuttgart, 1991).

The higher the particle size of an active ingredient, the more difficult is it to achieve a uniform content of the active ingredient in the tablets. The content uniformity can be determined according to international pharmacopeias as the relative standard deviation of the content of a sample of tablets. Values below 2% meet pharmaceutical requirements in terms of the homogeneity of active ingredient distribution and ensure good industrial processability of the pharmaceutical composition. The pharmacopeias, however, accept a standard deviation of up to 6.2%, which in practice could lead to production difficulties. Other embodiments of the invention, with a number of daily dosage units of 28 or a multiple of 28, for example two or three times 28, which each contain 2.0 mg or less of dienogest and less than 0.030 mg of ethinyl estradiol, are possible.

The peroral medication can also be used in hormone replacement therapy.

Other embodiments with gestagens, for example levonorgestrel, gestoden and others, and/or estrogens, such as estradiol and estradiol valerate, which alone or in combination are suitable for contraception as well as for hormone substitution (even sequentially) are possible. We have found that the active ingredient combination in the peroral medication of the invention also exhibits anti-androgenic properties besides contraceptive properties and hence can be used for prophylaxis and therapy of androgen-induced disorders, particularly acne.

### BRIEF DESCRIPTION OF THE DRAWING

The present invention will now be described in greater detail with reference to the attached drawing in which

FIG. 1 is a graphical illustration showing the time dependence of in vitro release of dienogest according to examples 1 to 3, and

FIG. 2 is a graphical illustration showing the time dependence of crystalline dienogest concentrations in human plasma obtained by simulation in silico.

### DETAILED DESCRIPTION OF THE INVENTION

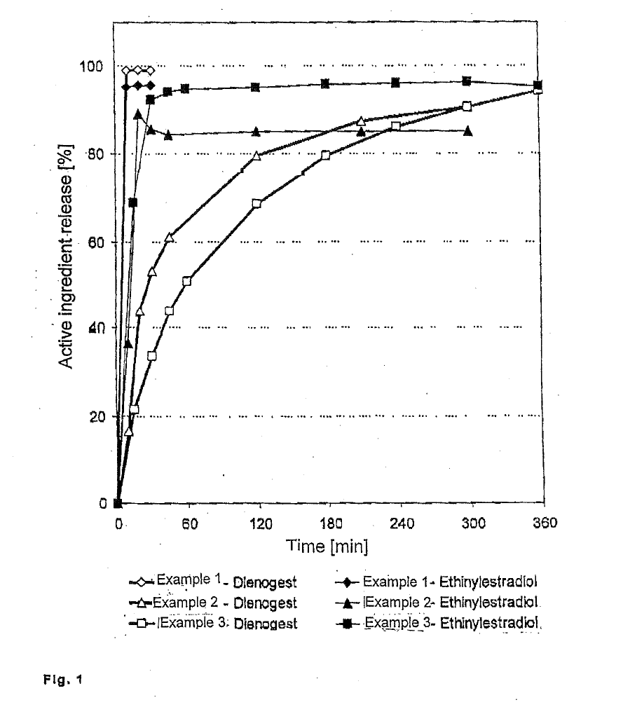

FIG. 1 shows the time dependence for in vitro release of dienogest and ethinyl estradiol as per examples 1 to 3. Example 1 describes the measurement of the time dependence of the active ingredient release of dienogest and of the active ingredient release of ethinyl estradiol, prepared and measured according to example 1, for a conventional sugar-coated tablet containing 2 mg of dienogest and 0.030 mg of ethinyl estradiol with a dienogest particle size of 3 μm. Example 1 is a comparative example describing the release profiles of active ingredients from a prior art contraceptive tablet. Example 2 describes the measurement of the time dependence of active ingredient release of dienogest and of active release of ethinyl estradiol, prepared and measured according to example 2 for a tablet containing 1.5 mg of dienogest and 0.015 mg of ethinyl estradiol with a dienogest particle size of 40 μm. Example 3 describes the measurement of the time dependence of the active ingredient release of dienogest and of the active ingredient release of ethinyl estradiol, prepared and measured according to example 3, for a coated tablet containing 1.5 mg of dienogest and 0.015 mg of ethinyl estradiol with a dienogest particle size of 65 μm.

Surprisingly, it was found that an increase in the particle size of the dienogest tends to slow down in vitro the fast dissolution of the active compounds from the composition. By fast release is meant a release of at least 70% of the active ingredient within about 30 minutes and preferably of at least 80% in about 20 minutes.

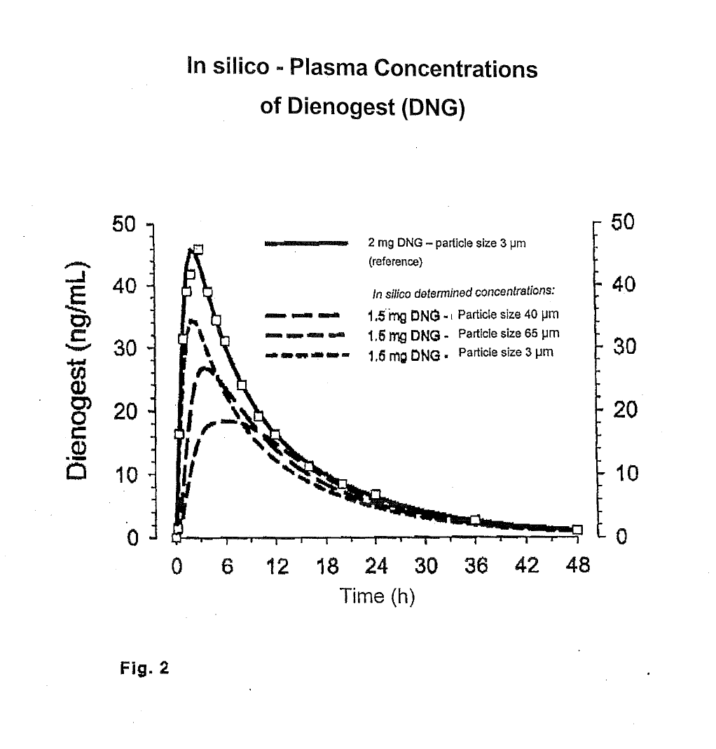

Based on measured human plasma concentrations after oral administration of dienogest (dosage 2 mg, particle size 3 μm), the plasma concentrations were simulated in silico (FIG. 2) and curves showing the time dependent release were plotted for different dosages with the same particle size and also for equal dosages with different particle sizes.

As can be seen, the maximum plasma concentration Cmax of micronized dienogest (particle size 3 μm) is reached after two hours independently from the dosage of dienogest that is administered. Moreover, it can also be seen that the greater the particle size at the same dosage of dienogest, the lower is the Cmax without reduction in the bioavailability. The bioavailability of dienogest can be obtained from the area under the curve (AUC) for the plasma concentration. At the same dosage of dienogest but with an increasing particle size, Cmax is nearly halved from 100% (3 μm) to 78% (40 μm) and 54% (65 μm). At the same time, with increasing particle size tmax the time to reach Cmax increases from 2 h at 3 μm to 3.5 h at 40 μm and finally to 6 h at 65 μm. After about 12 h, the absorption process even for dienogest with the maximum particle size of 65 μm is completed. Thereafter, independently of the particle size, dienogest is eliminated from the plasma at the same rate and, hence, the plasma concentrations are similar.

### PRACTICAL EXAMPLES

### Example 1

Valette is a conventional sugar-coated tablet for oral contraception containing 0.030 mg of ethinyl estradiol and 2.0 mg of dienogest in a tablet core covered with a sugar-containing coating.

Being a difficultly soluble active ingredient dienogest is usually micronized and used at an average particle size of about 3 μm. With this particle size distribution, the optimum dosage for reliable ovulation inhibition was found to be 2 mg of dienogest.

During the granulation an ethanolic solution of the ethinyl estradiol was sprayed in.

Tablets having the following composition were prepared:

Core:

| | |
|---|---|
| Dienogest | 2.000 mg |
| Ethinyl estradiol | 0.030 mg |
| Lactose monohydrate | 28.720 mg |
| Corn starch | 15.000 mg |
| Maltodextrin | 3.750 mg |
| Magnesium stearate | 0.500 mg |

All substances were granulated and mixed in a suitable manner and then pressed to form tablet cores having a diameter of 5 mm and weighing 50 mg.

Coating:

| | |
|---|---|
| Sucrose | 23.69340 mg |
| Glucose syrup | 1.65000 mg |
| Calcium carbonate | 2.40000 mg |
| Povidone K25 | 0.15000 mg |
| Macrogol 35.000 | 1.35000 mg |
| Titanium dioxide | 0.74244 mg |
| Carnauba wax | 0.01416 mg |

The substances for the coating were dispersed in water and sprayed onto the tablets by appropriate equipment until the resulting sugar-containing tablet had a final weight of 80 mg.

The tablets were examined for their active ingredient release by the method described in the European Pharmacopeia, 4th edition, original issue 2002, 2.9.3.

The conditions were as follows:

| | |
|---|---|
| Medium: | 1000 mL of water |
| Temperature: | 37° C. |
| Apparatus: | blade agitator, 50 rpm |
| Sampling: | 7 mL with replenishment |
| Analysis: | HPLC |

The following in vitro active ingredient releases were measured:

| | |
|---|---|
| 10 min | 99.0% of dienogest; 95.2% of ethinyl estradiol |
| 20 min | 99.1% of dienogest; 95.5% of ethinyl estradiol |
| 30 min | 99.0% of dienogest; 95.5% of ethinyl estradiol |

The uniformity of the dienogest content of the tablets was determined according to European Pharmacopeia 2.9.40. The relative standard deviation for the dienogest content of the tablets was 0.7%.

### Example 2

Determination and dissolution and blood level curves for a dienogest/ethinyl estradiol coated tablet (1.5 mg of dienogest=DNG/0.015 mg of ethinyl estradiol=EE) with the dienogest in the core of the coated tablet having an average particle size of 40 μm.

Dienogest with a particle size distribution median of 40 μm was used. This value was measured by laser light diffraction. The ethinyl estradiol in the form of an ethanolic solution was sprayed on during the granulation.

Tablets having the following composition were prepared:

Core:

| | |
|---|---|
| Dienogest | 1.500 mg |
| Ethinyl estradiol | 0.015 mg |
| Lactose monohydrate | 53.835 mg |
| Corn starch | 27.000 mg |
| Maltodextrin | 6.750 mg |
| Magnesium stearate | 0.900 mg |

All substances were granulated and mixed in a suitable manner and then pressed to form tablet cores having a diameter of 5.5 mm and weighing 90 mg.

Coating:

| | |
|---|---|
| Methocel | 3.000 mg |
| Macrogol | 0.600 mg |
| Talc | 0.600 mg |
| Titanium dioxide | 1.700 mg |
| Iron oxide pigment, red | 0.100 mg |

The substances for the coating were dispersed in water and sprayed onto the tablet cores by appropriate equipment until a coated tablet had a final weight of 96 mg.

The tablets were examined for their active ingredient release by the method described in European Pharmacopeia 2.9.3.

The conditions were as follows:

| | |
|---|---|
| Medium: | 1000 mL of water |
| Temperature: | 37° C. |
| Apparatus: | blade agitator, 50 rpm |
| Sampling: | 7 mL with replenishment |
| Analysis: | HPLC |

The following in vitro active ingredient releases were measured:

| | |
|---|---|
| 30 min | 53.3% of dienogest; 85.6% of ethinyl estradiol |
| 120 min | 79.5% of dienogest; 85.0% of ethinyl estradiol |
| 300 min | 90.5% of dienogest; 85.02% of ethinyl estradiol |

The uniformity of the dienogest content of the tablets was determined according to European Pharmacopeia 2.9.40. The relative standard deviation of the dienogest content of the tablets was 3.3%.

### Example 3

Determination and dissolution and blood level curves for a dienogest/ethinyl estradiol coated tablet (1.5 mg of dienogest=DNG/0.015 mg of ethinyl estradiol=EE) with the dienogest in the core of the coated tablet having an average particle size of 65 μm.

Dienogest with a particle size distribution median of 65 μm was used. This value was measured by laser light diffraction. Ethinyl estradiol in the form of an ethanolic solution was sprayed in during the granulation.

Coated tablets having the following composition were prepared:

Core:

| | |
|---|---|
| Dienogest | 1.500 mg |
| Ethinyl estradiol | 0.015 mg |
| Lactose monohydrate | 53.835 mg |
| Corn starch | 27.000 mg |
| Maltodextrin | 6.750 mg |
| Magnesium stearate | 0.900 mg |

All substances were granulated and mixed in a suitable manner and then pressed to form tablet cores having a diameter of 5.5 mm and weighing 90 mg.

Coating:

| | |
|---|---|
| Methocel | 3.000 mg |
| Macrogol | 0.600 mg |
| Talc | 0.600 mg |
| Titanium dioxide | 1.700 mg |
| Iron oxide pigment, red | 0.100 mg |

The substances were dispersed in water and sprayed onto the tablets by use of appropriate equipment until a coated tablet had a final weight of 96 mg.

The tablets were examined for their active ingredient release by the method described in European Pharmacopeia 2.9.3.

The conditions were as follows:

| | |
|---|---|
| Medium: | 1000 mL of water |
| Temperature: | 37° C. |
| Apparatus: | blade agitator, 50 rpm |
| Sampling: | 7 mL with replenishment |
| Analysis: | HPLC |

The following in vitro active ingredient releases were measured:

| | |
|---|---|
| 30 min | 33.6% of dienogest; 92.3% of ethinyl estradiol |
| 120 min | 68.6% of dienogest; 95.1% of ethinyl estradiol |
| 300 min | 90.6% of dienogest; 96.3% of ethinyl estradiol |
| 360 min | 94.3% of dienogest; 95.2% of ethinyl estradiol |

The uniformity of the dienogest content of the tablets was determined according to European Pharmacopeia 2.9.40. The relative standard deviation of the dienogest content of the tablets was 2.2%.

Preferably the effective ingredients in the peroral medication for preventing conception consist only of crystalline dienogest and ethinyl estradiol.

While the invention has been illustrated and described as embodied in a low-dosage peroral medication for contraception containing crystalline dienogest and ethinyl estradiol, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A peroral medication for contraception containing 2.0 mg or less of crystalline 17α-cyanomethyl-17β-hydroxyestra-4,9-dien-3-one and less than 0.030 mg of 17α-ethinyl estradiol as effective active ingredients for preventing conception, wherein said crystalline 17α-cyanomethyl-17β-hydroxy-estra-4,9-dien-3-one has an average particle size from 25 to 270 μm.

2. The peroral medication as defined in claim 1, wherein said average particle size is from 30 to 70 μm.

3. The peroral medication as defined in claim 1, wherein said average particle size is from 40 to 65 μm.

4. The peroral medication as defined in claim 1, containing from 0.75 to 2.0 mg of said crystalline 17α-cyanomethyl-17β-hydroxyestra-4,9-dien-3-one and from 0.015 to 0.020 mg of said ethinyl estradiol.

5. The peroral medication as defined in claim 1, containing from 1.5 to 2.0 mg of said crystalline 17α-cyanomethyl-17β-hydroxyestra-4,9-dien-3-one dienogest and from 0.015 to 0.020 mg of said ethinyl estradiol.

6. The peroral medication as defined in claim 1, comprising 21, 22, 23, 24, 25, or 26 daily dosage units containing said crystalline 17α-cyanomethyl-17β-hydroxyestra-4,9-dien-3-one and said ethinyl estradiol and 7, 6, 5, 4, 3, or 2 daily dosage units respectively not containing said crystalline 17α-cyanomethyl-17β-hydroxyestra-4,9-dien-3-one and not containing said ethinyl estradiol for a 28-day menstrual cycle.

7. A peroral medication for contraception comprising a tablet or a coated tablet for daily oral administration, wherein said tablet or said coated tablet contains an active ingredient combination effective for preventing conception, together with pharmaceutically acceptable carriers;
wherein said active ingredient combination consists of from 0.75 to 2.0 mg of crystalline 17α-cyanomethyl-17β-hydroxyestra-4,9-dien-3-one and from 0.015 to 0.020 mg of ethinyl estradiol; and
wherein said crystalline 17α-cyanomethyl-17β-hydroxy-estra-4,9-dien-3-one consists of crystals of said 17α-cyanomethyl-17β-hydroxy-estra-4,9-dien-3-one with an average particle size of from 25 to 270 μm.

8. A contraceptive preparation for oral administration consisting of 21 daily dosage units each consisting of an effective ingredient combination for contraception and 7 daily dosage units each not containing active ingredients for preventing conception, or consisting of 22 daily dosage units each consisting of said effective ingredient combination
for contraception and 6 daily dosage units each not containing said active ingredients for preventing conception, or
consisting of 23 daily dosage units each consisting of said effective ingredient combination for contraception and 5 daily dosage units each not containing said active ingredients for preventing conception, or
consisting of 24 daily dosage units each consisting of said effective ingredient combination for contraception and 4 daily dosage units each not containing said active ingredients for preventing conception, or
consisting of 25 daily dosage units each consisting of said effective ingredient combination for contraception and 3 daily dosage units each not containing said active ingredients for preventing conception, or
consisting of 26 daily dosage units each consisting of said effective ingredient combination for contraception and 2 daily dosage units each not containing said active ingredients for preventing conception;
wherein said effective ingredient combination for contraception consists of from 0.75 to 2.0 mg of said crystalline 17α-cyanomethyl-17β-hydroxyestra-4,9-dien-3-one and from 0.015 to 0.020 mg of said ethinyl estradiol; and
wherein said crystalline 17α-cyanomethyl-17β-hydroxy-estra-4,9-dien-3-one consists of crystals of said 17α-cyanomethyl-17β-hydroxy-estra-4,9-dien-3-one with an average particle size of from 25 to 270 μm.
